(19) United States
(12) Patent Application Publication
(10) Pub. No.: US 2008/0039896 A1
(43) Pub. Date: Feb. 14, 2008

(54) MEDICAL ELECTRODE WITH AT LEAST TWO POLES AND A PLUG

(76) Inventor: Peter Osypka, Rheinfelden-Herten (DE)

Correspondence Address: VOLPE AND KOENIG, P.C., UNITED PLAZA, SUITE 1600, 30 SOUTH 17TH STREET, PHILADELPHIA, PA, 19103, US

(21) Appl. No.: 11/834,766

(22) Filed: Aug. 7, 2007

(30) Foreign Application Priority Data
Aug. 9, 2006 (DE) 102006037122.4-54

(51) Int. Cl. A61N 1/04; A61N 1/36; A61N 1/39

(52) U.S. Cl. 607/5; 174/78; 607/9

(57) ABSTRACT

A medical electrode (1) with at least two poles arranged at its distal end (2) and preferably an anchor (3) as well as a plug (7) having a feeder (4) from the plug (7) to the poles is provided. The anchor (3), is formed at least from two in reference to each other insulated, parallel or in the same direction extending helixes (5) and (6), preferably having the same diameter. The proximal end of this feeder (4) and the helixes (5) and (6) forming it is connected to the plug (7) electrically conducting in the operational state, with one helix (5) being connected to a protrusion or cathode (9), that extends with a pin-shape opposite to a plug pin (8) that can be inserted into a medical device comprising a power source, which also continues to the plug pin (8). The other helix (6) is connected to a socket, bushing, or anode (10) allocated to the plug (7), insulated in reference to the plug pin and the cathode (9). The proximal ends of the helixes (5) and (6) are here connected to sockets (11) and (12), which on the one hand are connected or welded to the cathode (9) and on the other hand to the anode (10) in the operational state, so that the helixes (5) and (6) are not required to be welded to parts of the plugs (7).

### BACKGROUND

The invention relates to a medical electrode with at least two poles arranged at and/or near its distal end, namely at least one anode and one cathode, and with a plug, with the leads to the poles being formed from at least two helixes, insulated in reference to each other and extending particularly parallel or in the same direction, and with the proximal end of the leads and the helixes forming it being connected to the plug in the operational position, with one helix being connected to a pin-shaped arrangement or cathode, protruding opposite in reference to an insertable plug pin, which can be inserted into a medical device comprising a power source, and the other helix being connected to an insulated socket, bushing, or anode allocated to the plug and insulated in reference to the plug pin.

Such electrode arrangements with plugs are known in the medical field. Frequently such electrode-plug—arrangements are used in the context of implanted pacemakers, which is engaged via a plug in bipolar electrodes leading to the heart, in particular. Here, the electrode is provided with at least two helixes, generally extending in the same direction and insulated in reference to each other, in order to supply the anode and the cathode with power or high-voltage, in case the arrangement shall—serve for defibrillation as well. Here, arrangements with for example four helixes for a total of four poles are also possible.

An example of such a medical electrode with references to additional examples is described in DE 103 02 319 A1.

The connection of the helixes allocated to the different poles or electrodes, to the plug is difficult. Here it is known for one helix to be welded to a distal protrusion of the plug pin, serving as the cathode, which requires great care in order for a lasting connection to form, capable of withstanding the stress. A second helix can be arranged at a socket part, arranged at the plug and generally forming the anode, in order to create the connection to a second pole, which in the past also happened by way of welding and again requires great care.

Due to the fact that the entire arrangement must be tested, and this is only possible after the connection of the helixes to the plug, there is the risk that such an arrangement must be thrown out as defective when tests show that the connection has not been made successfully.

### SUMMARY

Thus, there is the object to provide an arrangement of the type mentioned at the outset, i.e. a medical electrode with several poles and a plug, in which the production is designed such that even less experienced persons with regard to the welding technology can create a secure connection, and the welded connections can be tested without having to reject the finished electrode provided with a plug, in case of a failed test.

In order to meet this seemingly contradicting task, the electrode defined at the outset is characterized in that the proximal end of at least the helix that can be connected to the pin-shaped arrangement or cathode is connected in a power conducting fashion to a socket fitting onto or into the pin-shaped arrangement or cathode and enclosing or engaging it in the operational position. In this way, at least the helix provided with a pole can be connected to a socket and then the conductivity of the connection is tested prior to completing the entire connection to a plug. Furthermore, such a socket can be connected much easier to the respective part of the plug than individual, generally very thin wires of a helix to an associated part of the plug, for example by way of welding, so that the production with sufficient safety from defects is possible even for less experienced persons.

Here, it is particularly beneficial when the proximal ends of two or all helixes, connected to the plug in the operational position, are electrically conducting and connected to sockets spaced apart from each other in the radial and/or axial direction, with one of them matching the cathode and the other matching the anode of the plug in an electrically conducting fashion. Therefore, the helixes can first be connected to sockets, which subsequently can be plugged together and connected to the respective parts of the plug so that first the connections of the helixes to the sockets are tested and the connection to the plugs can only be performed after a positive test, with this connection of the plug to the sockets being simpler than a connection of the plug using the wires of the helixes. By the seemingly additional expense to arrange sockets at the proximal ends of the helixes, the testing is facilitated and the risk of rejections is reduced and additionally the assembly is facilitated. When here the cathode at the plug is embodied tubular or socket-shaped, the matching “socket” could be inserted into this cathode for the final assembly and inside it could be accessible via a penetration for mounting, thus this “socket” then has a solid cross-section as well.

In a preferred embodiment of the electrode according to the invention, the sockets arranged at the proximal end of one or more helixes can be connected to the helixes. This allows easy testing before the final connection to the plug is made, which in turn is relatively easy and thus is also successful when less experienced persons perform the final plug connection.

The sockets may have different diameters or cross-sections and engage the plug in the operational position, arranged concentrically in reference to each other. In this way, it is considered that the cathode and the anode generally have different diameters in such a plug in order to keep the overall length of the plug within limits.

Here the helixes of the feeders insulated in reference to each other may have the same diameter, and at least the helix connected to the larger socket in the operational position, in particular the anode, may be widened at its proximal end to the size of the larger socket. Due to the fact that the wires forming the helixes are relatively flexible or well bendable, such a widening of one helix is easily possible for fastening to a socket with a larger diameter.

The diameter of the smaller socket can correspond to the respective helix so that it requires no adjustment for fastening to the smaller socket, which may be possible, though, if the smaller socket also requires a cross-section deviating from the dimensions of the corresponding helix.

At least one socket can be welded to the preferably pin-shaped cathode and/or the preferably socket-shaped anode of the plug in the operational state, for example by spot-welding or laser welding. This not only results in a durable and mechanically solid connection, but also one which conducts electricity particularly well. Here, the welding of a socket to a matching counterpart is relatively easy.

An embodiment of the invention with particularly advantageous importance standing alone, can provide that at least one of the fastening sockets arranged at the proximal end of a helix has a recess at its exterior or interior for accepting at least a portion of the cross-section of the end of the insulated helix to be connected thereto, and that the end of the helix engaging said recess is welded to the socket, in particular laser welded. This results in a predetermined position for fastening the helix to the socket, which allows an improved connection

Here it is particularly beneficial when the recess at the socket or the sockets extends helically, and the direction of the incline of the helical line of the recess corresponds to a progression of the helical line of the helix to be connected thereto. In this way, a type of a screwed connection can be practically created between the helix and the socket, which is then secured by a mutual welding and is made stress-resistant. Here, the helical form of the recess results in a correspondingly large and long contact between the socket and the helix over a relatively low axial extension or length of the socket and thus also in a respectively effective mutual connection and/or welding. The welded connection between the recess of the socket and the helix of the feeder can here be provided at both edges of the recess. When such a helix is welded to a socket without a recess, a welding seam develops practically only along the linear contact of the helix to the socket. By the beneficial recess, into which the helix engages at least partially with its cross-section, along the edges of the recess at two lines of the helix, a welding seam can be provided along each line, which improves the mutual connection and the electrical conductivity.

The recess, beneficially in the surface of the socket, can be formed penetrating the entire wall thickness of the socket to form a hole or slot. Here, the recess may for example have a groove-shaped cross-section to adjust to the cross-section of the wires forming helixes, and if necessary, extend additionally through the entire wall thickness of the socket, in order for the helixes to protrude as little as possible or not at all beyond the surface of the socket. By the adjustment of the cross-section of the recess to the helical wires, in turn the mutual form-fitting and the mutual connection are improved. However, the production of a socket with a penetrating slot of the same thickness and width is easier.

The two helixes provided for different poles of the electrode arrangement can beneficially be provided each with a socket having helically shaped or perhaps groove-shaped recesses and/or recess them penetrating the entire wall thickness of the socket to connect to one end of the corresponding helix. Thus, two or all poles of an electrode arrangement according to the invention can be reproduced in the same manner, in particular, having sockets and here being particularly well and lastingly welded via the helical recesses so that subsequently only these sockets need to be connected and welded to the respective parts or areas of the plug.

After placement, the smaller socket can be first welded pin-shaped to the plug at its distal area, subsequently the larger socket is moved over it in the axial direction using the elasticity of the helix connected thereto and can be connected to the anode arranged coaxially at the outside.

Here it may be beneficial if the socket of the cathode has at least one slot at its proximal face for mounting the welding site. This way a relatively long welded seam can be provided without any distortion at the site, namely in the area of this slot, while without any slots welding along the face had to be sufficient.

In order for the anode itself to have a sufficiently large axial extension it may be beneficial when the anode of the plug has at least one penetration and the welded connection of the cathode is arranged radially below or behind said penetration. In this way, first the smaller socket of the cathode connection can be pushed onto the plug cathode, until it has reached the penetration of the anode, and subsequently it can be welded to the cathode through this penetration. Then, the larger socket can again be displaced over the penetration and in turn be welded.

The electrode arrangement according to the invention can be used in a particularly useful and advantageous manner as a bipolar stimulation device or pacemaker or as a defibrillator electrode, i.e. the plug can for example be adjusted to a pacemaker or defibrillator or a similar medical device that can be implanted and be connected to the electrode feeder to the heart in the manner according to the invention.

Primarily in combinations of the above-described individual or several features and measures, a connection of the medical electrode to its corresponding plug develops meeting the requirements in solidity, durability, and electrical conductivity, with it being possible in an advantageous manner first to test the electric conductivity before the final fastening at the plug occurs so that not only the assembly is facilitated but also the risk of rejections is considerably reduced.

An additionally beneficial embodiment of the arrangement according to the invention may be provided in that in the operational position, an insulating and centering socket is arranged or plugged in between at least sectionally concentric sockets, i.e. overlapping each other in the axial direction over at least a portion of their axial dimensions, an insulation and centering socket. In this way, the sockets are also fixed in the radial direction in reference to each other and the risk of a potential power surge or short is further reduced.

### BRIEF DESCRIPTION OF THE DRAWINGS

In the following exemplary embodiments of the invention are described in greater detail using the drawings. They show in a partially schematic illustration:

FIG. 1 a side view of a medical electrode with poles arranged at its distal end and an anchoring, by which the electrode can be fixed inside the heart for example as well as a plug arranged at the proximal end and connected to the electrode feeder for connecting a medical device comprising a power source, such as for example a pacemaker,

FIG. 2 an enlarged scale view, at the proximal end of the two helixes forming the electrode feeder, insulated from each other and extending parallel and in the same direction, with one helix being widened in reference to its last windings as well as two sockets provided with helical recesses, with their diameter or cross-section matching the ends of the helixes,

FIG. 3 an illustration according to FIG. 2 after the connection of the proximal ends of the helixes with their matching sockets, with the last windings of the helixes engaging the helical or screw-like arranged recesses of the sockets and being welded thereto,

FIG. 4 a side view of the plug with a cathode arranged thereon, protruding pin-shaped at the distal end, and an anode concentric in reference thereto as well as the proximal end of the feeder according to FIG. 3 prior to the mutual fastening,

FIG. 5 a view according to FIG. 4 after the smaller socket is pushed onto the cathode and the fastening of said socket by way of laser welding, prior to the coaxial displacement of the larger socket onto the anode of the plug,

FIG. 6 an illustration according to FIG. 5 after the placement of the larger socket onto the anode of the plug and its fastening thereat via laser welding,

FIG. 7 a longitudinal cross-sectional view of the arrangement according to FIG. 6 according to a line A-A in FIG. 6,

FIG. 8 a view according to FIG. 4 in a slightly modified arrangement, in which the smaller socket has a recess or slot at its proximal face,

FIG. 9 a view according to FIG. 5, with the laser welding being performed at the recess of the smaller socket, before the larger socket serving as the anode is displaced similar to FIGS. 6 and 7 and mounted to the plug,

FIG. 10 a view according to FIGS. 4 and 8, with the anode socket of the plug having a penetration at the location where the smaller socket contacts the electrode feeder in the operational position,

FIG. 11 a view according to FIGS. 5 and 9 after the fastening of the smaller socket to the cathode of the plug, before the axial displacement and fastening of the larger socket to the anode of the plug,

FIG. 12 a view of a modified embodiment in which the anode of the plug has a larger penetration and/or only forms a partial socket after the fastening of the smaller or cathode socket to the cathode of the plug, prior to fastening the larger socket to the anode,

FIG. 13 a longitudinal cross-sectional view of the arrangement according to FIG. 12 along the section A-A in FIG. 12,

FIG. 14 an enlarged scale view of an arrangement according to FIG. 3, with additionally an insulating socket being provided between the small and the large socket before the placement.

FIG. 15 a side view of the plug and its connection to the feeder provided with two helixes via the smaller and the larger socket in a lateral view,

FIG. 16 a longitudinal cross-sectional view of the arrangement of FIG. 15 along the section A-A in FIG. 15, with the insulating socket shown separately in FIG. 14 being arranged between the cathode socket and the anode socket isolating them from each other, as well as,

FIG. 17 a somewhat reduced scale view of the arrangement according to FIG. 15, for example, thus the fastening of the plug pin to the sockets provided with the electrode feeders prior to the placement of an insulating socket onto the anode of the plug.

### DETAILED DESCRIPTION OF THE PREFERRED EMBODIMENTS

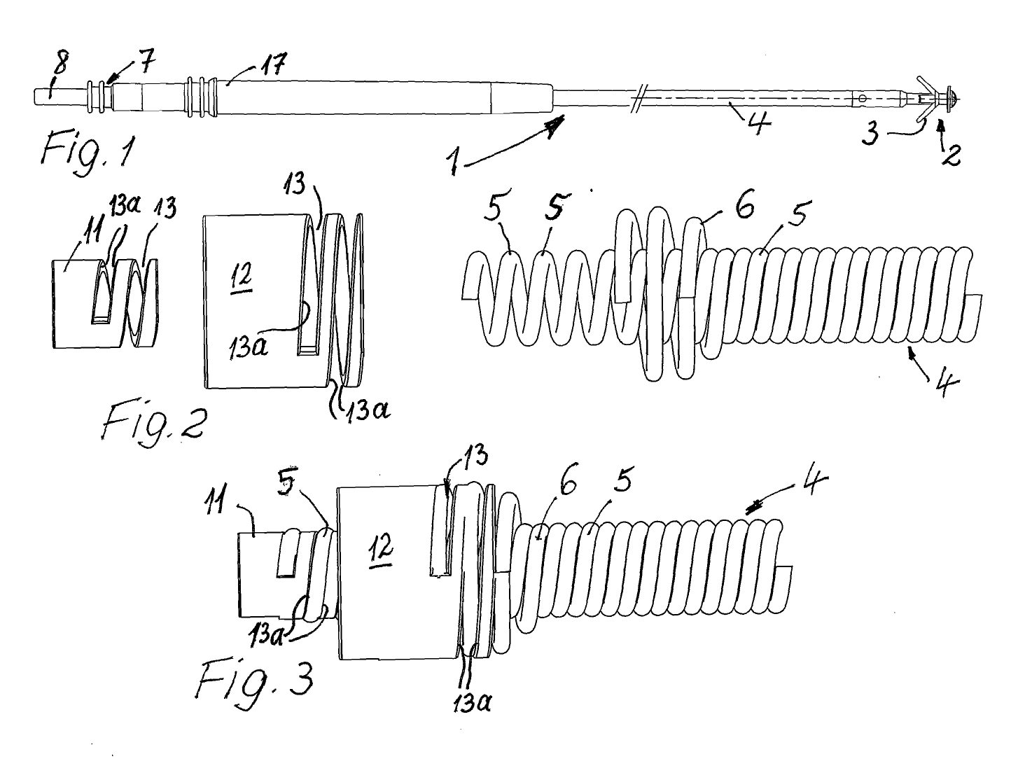

A medical electrode called 1 in its entirety (FIG. 1) has at its distal end 2 two poles in a manner known per se, i.e. an anode and a cathode and additionally an anchor 3 for fixing it inside a heart in the heart connective tissue, for example.

A feeder 4 for these poles and the anchor 3, in the exemplary embodiment, according to FIGS. 2, 4, 7, 14, and 16, is formed from two parallel helixes 5 and 6, isolated from each other and extending in the same direction, each of which extending to one of the poles.

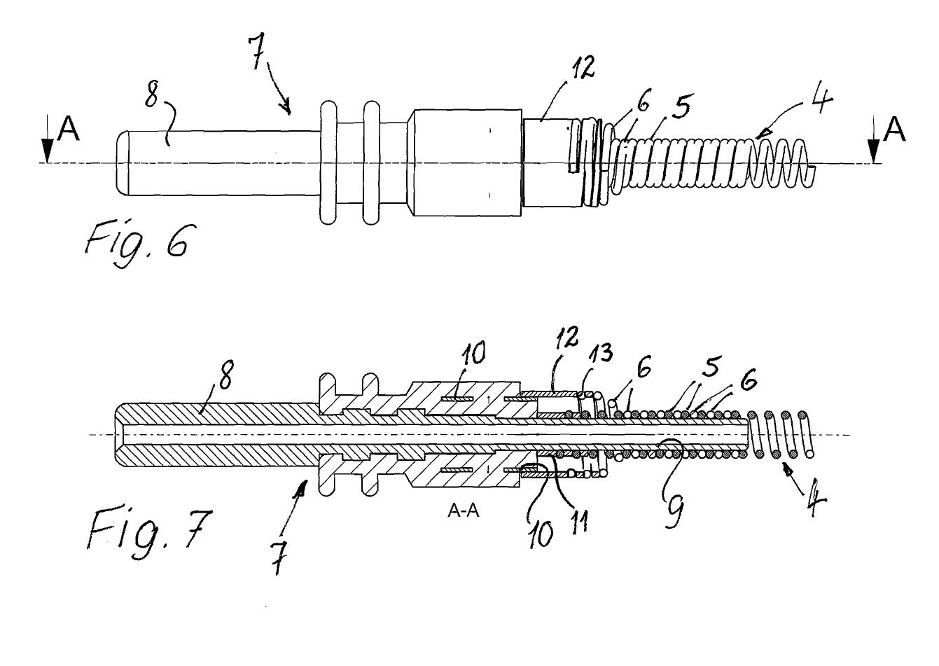

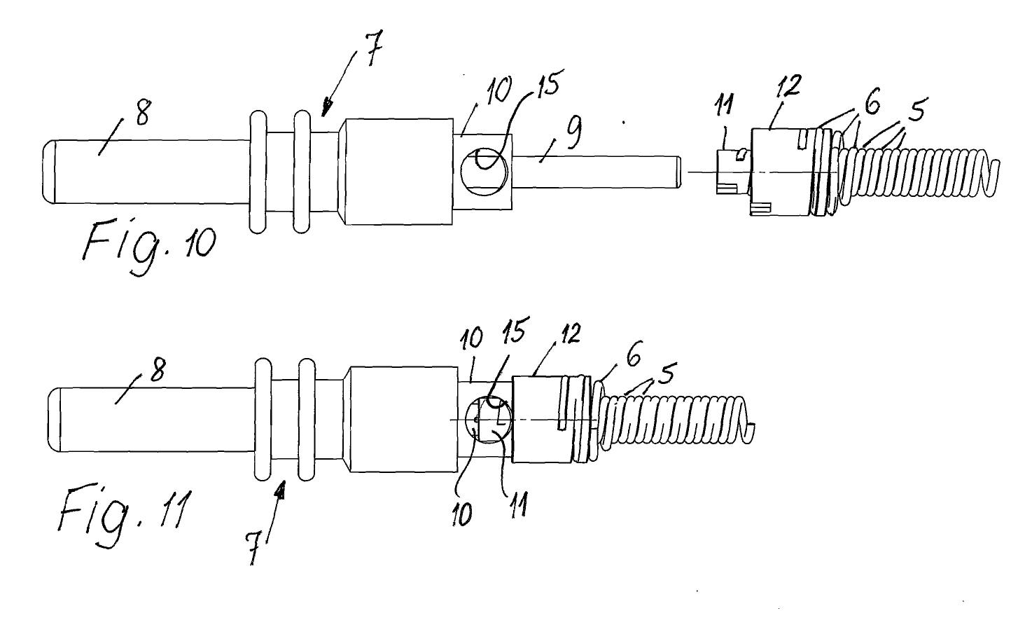

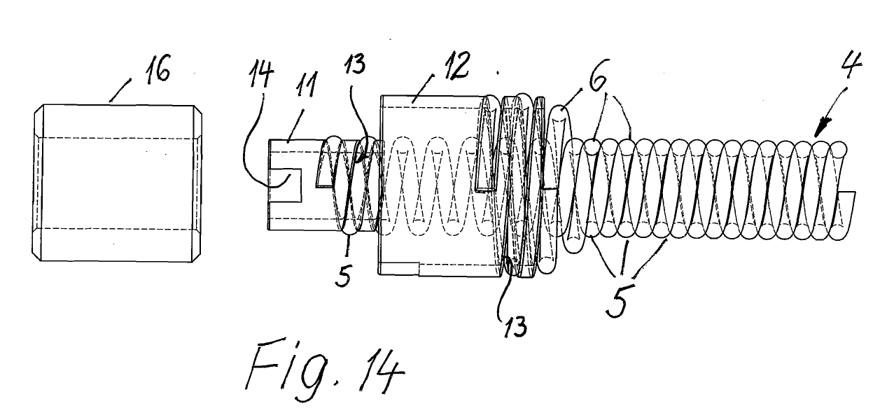

The proximal end of this feeder 4, which is shown in FIGS. 2 through 16 in a manner described below and the helixes 5 and 6 forming them are connected in the operational position to a plug 7 in a manner described below, with one helix 5 being connected to the plug pin 8 of the plug 7, which can be inserted into a medical device comprising a power source, having an opposite protruding pin-shaped arrangement or the cathode 9 (cf. e.g., FIG. 4) and the other helix 6 being connected in the operational position (FIG. 7 or 16) to a socket, bushing, or anode 10 insulated in reference to the plug pin 8 and the cathode 9 and allocated to the plug 7.

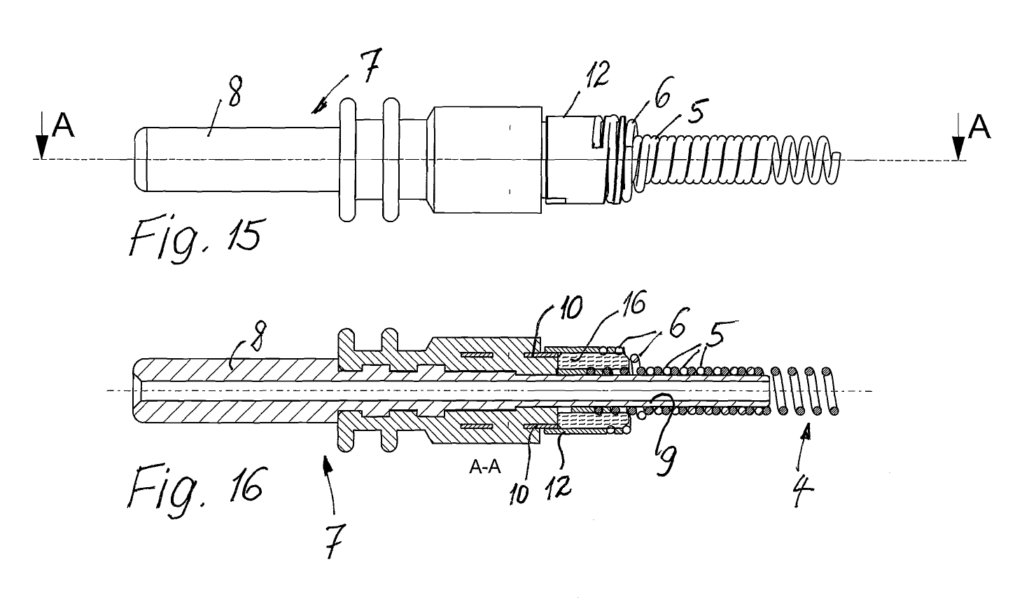

Here in several figures, it is discernible and particularly obvious in FIGS. 2 and 14, that the proximal end of the helix 5, which can be connected to the pin-shaped arrangement or cathode 9, i.e. with the plug cathode 9 being connected to a socket 11 fitting on the cathode 8 and enclosing it electrically conducting in the operational state according to FIGS. 4, 5, 7, 13, and 16, with its diameter being equivalent to the corresponding helix 5.

In the already mentioned Figures, it is additionally discernible that the helix 6 is connected to the proximal end of the plug 9 of the anode 10 in the operational position is also connected to the socket 12, which is spaced apart from the first-mentioned socket 11 in the radial direction because it has a larger diameter which matches the respectively large diameter of the anode 10. This is clearly discernible in FIGS. 7 and 16, in particular.

The sockets 11 and 12 have therefore different diameters or cross-sections and are arranged approximately concentric in reference to each other in the operational position when they engage the plug 9 and/or are connected thereto.

The helixes 5 and 6 of the feeder 4 are insulated from each other and have the same diameter in the exemplary embodiments, thus they are located side-by-side in the axial direction. The helix 6 connected to the larger socket 12 and thus to the anode 10 of the plug 9 in the operational position is here widened at its proximal end to the larger size of the larger socket 12, as particularly well discernible in FIGS. 2 and 3 as well as in FIG. 14 and additional Figures.

In FIGS. 3, 4, 8, 10, 14, and 15 as well as the other Figures it is indicated that the sockets 11 and 12 arranged at the proximal end of the helixes 5, and 6 are connected to these helixes in a preproduced fashion, which considerably facilitates the assembly of the plug 7 in reference to an arrangement in which these helixes 5 and 6 have to be welded directly to the plug parts 9 and 10.

The sockets 11 and 12 can in turn be welded in the operational condition to the cathode 9 and the anode 10 of the plug 7, for example by spot-welding or laser welding, with this welding being realized in a considerably simpler fashion than even by welding the helixes directly to the corresponding socket parts.

Primarily in FIG. 2, it is shown that the fastening sockets 11 and 12 to be mounted or arranged in the operational state to the proximal end of the helixes 5 and 6 at their outside or inside are provided with a recess 13 for accepting at least a portion of the cross-section of the blank or insulated end of the helix to be connected to the respective socket and that the blank ends that each engage these recesses 13 of the respective helixes 5 or 6 can be welded or laser welded to the corresponding socket 11 and 12, as indicated in FIG. 3 and other figures. Here, these recesses 13 extend at the two sockets 11 and 12 according to FIG. 2 in a helical shape, with the direction of the incline of the respective helical line corresponding to the recess 13 of the helical extension of the helix 5 or 6 to be connected thereto, which is particularly discernible in FIG. 3.

In this way, the welded connection between the recess 13 of the respective socket and the corresponding helix of the feeder 5 can be provided to the two edges 13a of the recess 13 which results in a better and tighter connection than helixes that were only welded to the exterior of the respective socket after the insulation, which also would be possible, though.

In the exemplary embodiments, the recesses 13 penetrate the entire wall thickness of the respective sockets 11 and 12 so that the helixes can be lowered to a certain extent into the socket wall or the socket casing and a particularly good form-fitting is achieved in the axial direction. In the exemplary embodiment, the recess 13 is therefore embodied as a recess or a helical slot.

The sockets 11 and 12 may have additional embodiments, as shown in individual exemplary embodiments.

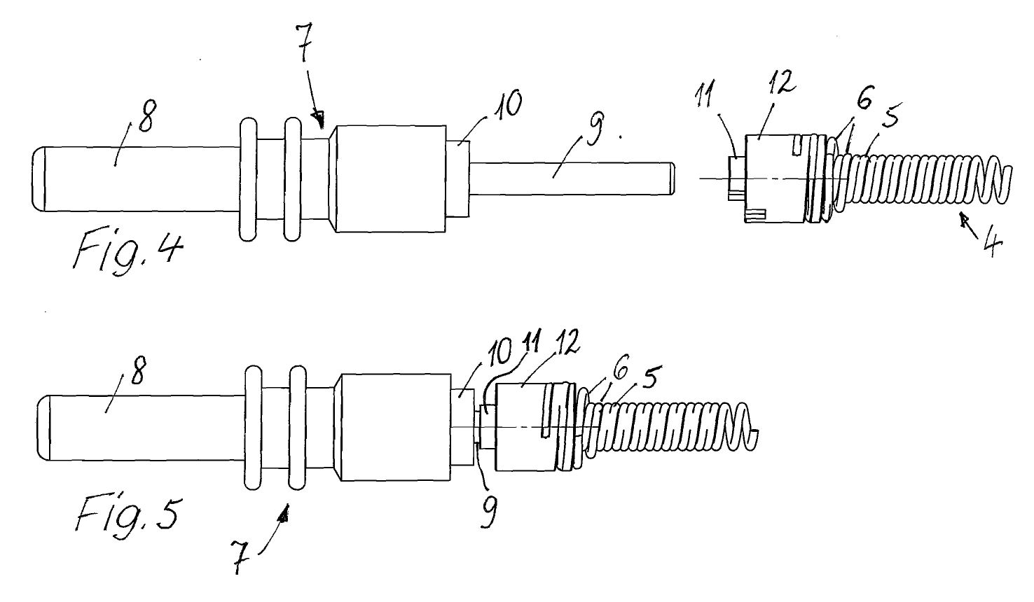

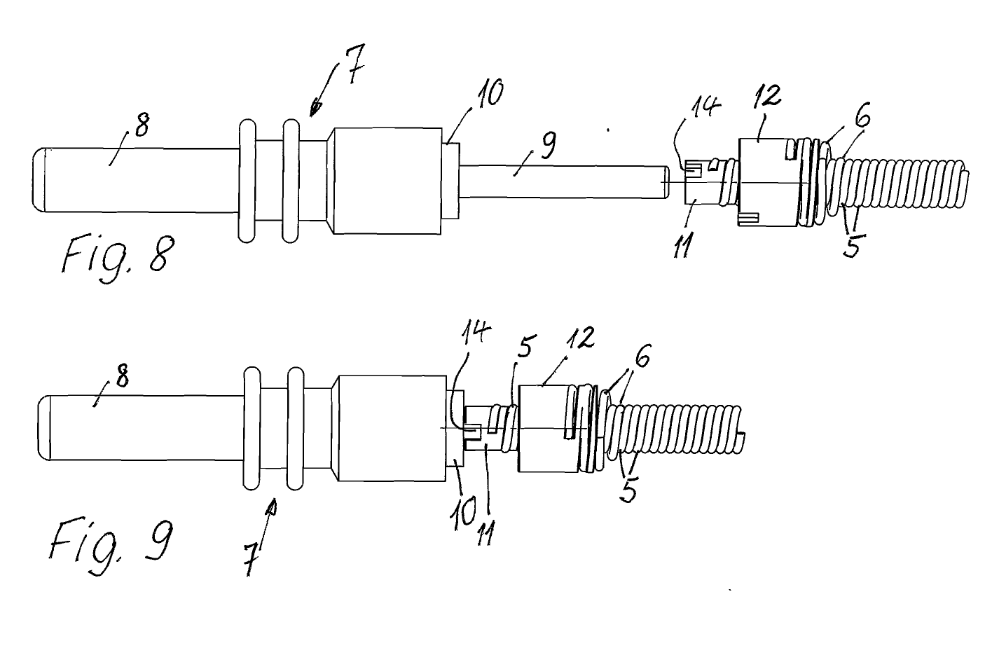

For example, the socket 11 of the cathode 9 can at its proximal face according to FIGS. 8, 9, 12, and 14 be provided with at least one slot 14 for mounting at the welding site to connect it to the cathode 9, which is primarily beneficial when this socket 11 reaches relatively far into the socket 7 and its face is hard to access for welding. It may also be welded to the cathode immediately at its face without such a deformation of the face according to FIGS. 3 and 5.

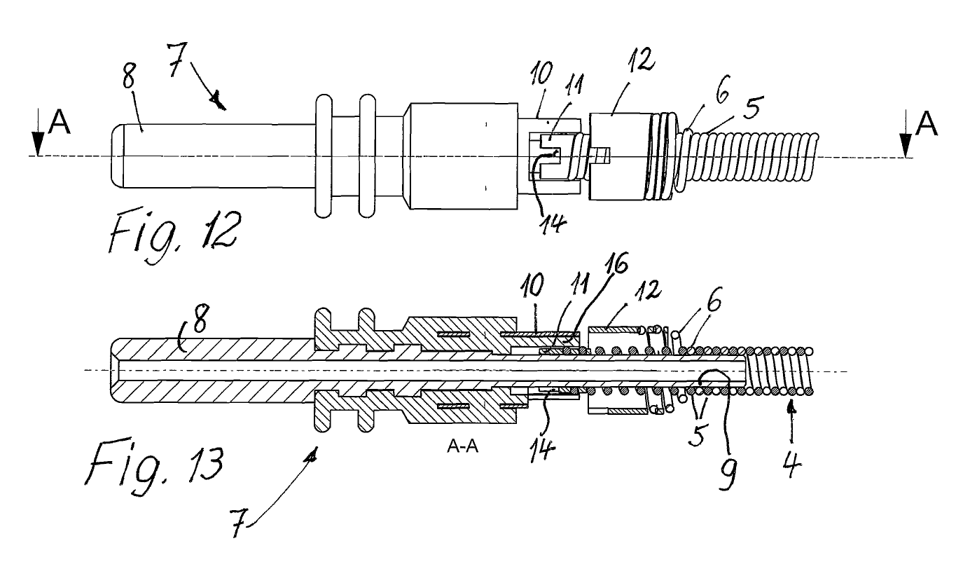

In another exemplary embodiment according to FIGS. 10 and 11, the space available for the anode 10 and the cathode 9 is so narrow that the anode 10 of the plug 7 has at least one penetration 15 and the welded connection of the cathode 9 is arranged radially below or behind this penetration 15.

Here it should be mentioned that in several illustrations the smaller socket 11 is connected to the cathode 9 of the plug 7, the larger socket 12 still remaining to be displaced axially in the proximal direction in order to reach its end position and operational state. Such illustrations are FIGS. 3, 5, 9, 11, 12, and 13.

Due to the elasticity of the helix 6 connected to the larger socket 12 for the anode 10, the larger socket 12 can be moved easily in the axial direction after the fastening of the smaller socket 11, placed onto the anode 10 and fastened there, as shown in FIGS. 6 and 7 as well as 15 and 16.

In an advantageous manner, in the operational position, an insulation or centering socket 16 is located between the concentric sockets 11 and 12, which according to FIG. 13, is made in one piece with the plug 7, according to FIGS. 14 and 16 embodied as a separate centering socket 16, which is inserted in the operational position between the two sockets 11 and 12. Using this insulating and centering socket 16, on the one hand, the assembly is facilitated and, on the other hand, the safety from power surges or shorts is improved.

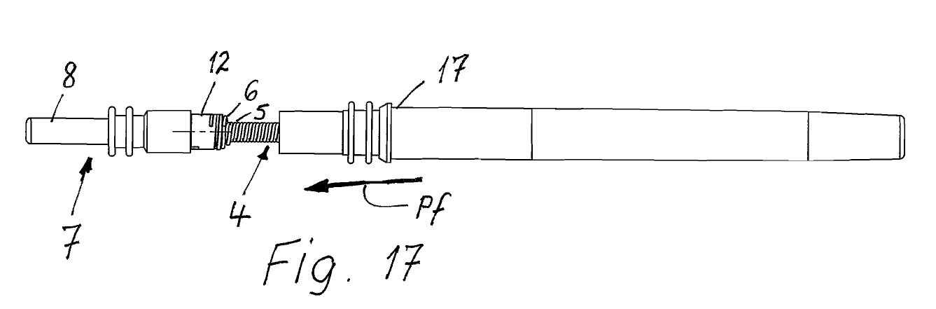

FIG. 17 also shows how the finished connection between the feeder 4 and the plug 7 can be covered by a grommet, with the grommet 17 in FIG. 17 being shown prior to the plug being plugged onto the connector and able to be displaced in the direction of the arrow Pf until the position discernible in FIG. 1 is reached.

The medical electrode 1 can be combined, in a manner known, to a pacemaker electrode or a stimulation or defibrillation electrode or an electrode combining several functions. In all these cases, on the one hand, the testing of the conductivity of the helixes insulated from each other is possible before the plug 7 is connected thereto, so that a greater amount of rejections can be avoided.

Furthermore, using the sockets 11 and 12, the connection and assembly to the plug 7 is facilitated and simultaneously improved.

The medical electrode 1 is at least formed with two poles, arranged at its distal end 2, and preferably with an anchor 3 as well as a plug 7 having a feeder 4 from the plug 7 to the poles and the anchor 3, which are formed from at least two helixes 5 and 6 isolated in reference to each other, extending parallel or in the same direction, preferably encircling an identical diameter. The proximal end of this feeder 4 and the helixes 5, and 6 forming it is connected in the operational state to the plug 7 in an electrically conducting manner, with the helix 5 being connected to a pin-shaped protrusion or a protruding opposite cathode 9, a plug pin 8 that can be inserted into a medical device comprising a power source, thus continuing to the plug pin 8, e.g., in one piece, and the other helix 7 being connected to the socket, bushing, or anode 10 corresponding to the plug 7 and isolated in reference to the plug pin and the cathode 9. The proximal end of the helixes 5 and 6 are here connected or welded to sockets 11 and 12, which on the one hand are connected or welded to the cathode 9 and on the other hand to the anode 10 in the operational position so that the helixes 5 and 7 do not require immediate welding to parts of the plug 7.

1. A medical electrode (1) comprising at least two poles with at least one of the poles being at or near a distal end (2) thereof, the at least two poles including at least one anode and one cathode, and having a plug (7) with a feeder (4) extending from the plug (7) to the poles comprising at least first and second helixes (5, 6) extending parallel or in the same direction and isolated from each other, and a proximal end of the feeder (4) and the at least first and second helixes (5, 6) forming the feeder being connected to the plug (7) in an operational state with the first helix (5) being connected via a first socket (11) to a pin-shaped arrangement or cathode (9) of the plug opposite to a protruding plug pin (8) that can be inserted into a medical device comprising a power source, and the second helix (6) being connected to a socket, bushing, or anode (10) allocated to the plug and being isolated relative to the plug pin (8) or the cathode (9), the first socket (11) connected to the first helix (5) fitting onto or into said pin-shaped arrangement or cathode (9) and enclosing or engaging it in an operational state in an electrically conducting manner.

2. An electrode according to claim 1, wherein the proximal end of the second helix (6) that is connected to the plug (7) in an electrically conducting manner in the operational position has a second socket (12), spaced apart from the first socket (11) in at least of a radial or axial direction, with one of the sockets corresponding to the cathode (9) and the other of the sockets corresponding to the anode (10) of the plug (7) for connection in an electrically conducting manner.

3. An electrode according to claim 2, wherein the first and second sockets (11, 12) arranged at the proximal end of the at least first and second helixes (5, 6) are connected to the helixes (5, 6).

4. An electrode according to claim 2, wherein the sockets (11, 12) have different cross-sections or diameters and engage the plug 7 in the operational state concentrically relative to each other.

5. An electrode according to claim 2, wherein the at least first and second helixes (5, 6) of the feeder (4) are isolated from each other and have the same diameter and at least the second helix (6) that is connected to the second socket (12), which is larger than the first socket (11), and corresponds to the anode, is widened at a proximal end thereof to a size of the second socket (12).

6. An electrode according to claim 5, wherein a diameter of the first socket (11) is equivalent to a diameter of the first helix (5).

7. An electrode according to claim 5, wherein at least one of the first and second sockets (11, 12) is connected to the pin-shaped cathode (9), which is connected to the pin plug (7) in one piece, or with a socket-shaped anode (10) of the plug (7) and are welded in the operational state.

8. An electrode according to claim 5, wherein at least one of the first and second sockets (11, 12) arranged at the proximal end of the at least first and second helixes (5, 6) is provided at an exterior or interior thereof with a recess (13) for accepting at least a portion of a cross-section of an end of a respective one of the at least first and second helixes (5, 6) to be connected to the respective socket (11, 12), and the end of the helix (5, 6) engages the recess (13) and is welded to the respective socket (11).

9. An electrode according to claim 8, wherein the recess (13) extends helically in the at least one of the first and second sockets (11, 12) and a direction of incline of a helical line of the recess (13) is equivalent to a helical progression of the helix (5, 6) to be connected thereto.

10. An electrode according to claim 9, wherein a welded connection is provided between the recess (13) of the at least one of the first and second sockets and the helix of the feeder (4) at both edges (13a) of the recess (13).

11. An electrode according to claim 10, wherein the recess (13) penetrates an entire wall thickness of the socket (11, 12) and is formed as a recess or slot.

12. An electrode according to claim 11, wherein the helixes (5, 6) are provided for different poles of the electrode arrangement and each of the at least first and second sockets (11, 12) are provided with helically-shaped recesses (13) for connecting to the ends of the corresponding first and second helixes (5, 6).

13. An electrode according to claim 12, wherein the first socket (11) for the cathode (9), at a proximal face, has at least a slot or a recess (14) for a welding site.

14. An electrode according to claim 13, wherein the anode (10) of the plug (7) has at least one penetration (15) and a welding connection of the cathode (9) is arranged radially below or behind the penetration (15).

15. An electrode according to claim 14, wherein in the operational state, an insulating and centering socket (16) is arranged concentrically between the first and second sockets (11, 12), which is provided in one piece with the plug (7) or is a separate part.

16. An electrode according to claim 1, wherein the electrode comprises a bipolar stimulation or pacemaker or defibrillator electrode.
